(19) United States
(12) Patent Application Publication
(10) Pub. No.: US 2014/0081361 A1
(43) Pub. Date: Mar. 20, 2014

(54) PERIORBITAL EDEMA REDUCTION DEVICE

(71) Applicant: Ishigaki Dhayan, East Sussex (GB)

(72) Inventor: Ishigaki Dhayan, East Sussex (GB)

(73) Assignee: KARSTARK LTD., East Sussex (GB)

(21) Appl. No.: 14/118,607

(22) PCT Filed: May 18, 2012
(86) PCT No.: PCT/IB2012/052514

§ 371 Date: Nov. 19, 2013

(30) Foreign Application Priority Data
May 20, 2011 (GB) 1108599.0

(51) Int. Cl. A61F 7/10

(52) U.S. Cl. CPC A61F 7/10
USPC 607/109

(57) ABSTRACT

A portable head-mounted device for cooling the under-eye area of skin, said device comprising at least one cooling element arranged to be located adjacent the skin of the under-eye area when the device is mounted to the head, the cooling element having a first side that faces the under-eye area of skin when the device is mounted to a head and a second side that faces away from the skin of the under-eye area; said at least one cooling element comprising: liquid retaining means adapted to retain a liquid provided thereto; and heat transfer means adapted to transfer heat by conduction to a liquid retained by the liquid retaining means to promote evaporation of said liquid thereby providing a cooling effect when the device is mounted to the head in use

The present invention relates to a portable head-mounted device for cooling under-eye area of skin.

Facial edema is a visual inflammation of the skin, and is commonly caused by a build-up of fluid in the sinuses under the skin. The area of skin beneath the eye is particularly susceptible to this condition, which can lead to the formation of “eye-bags”.

The causes of facial edema are numerous. It can be caused by a lack of sleep or a hereditary predisposition, but often it is a sign of health problems as a result of poor dietary habit. For example, dehydration caused by an excess of salt, sugar, alcohol and fats result in water retention in the facial tissue, leading to edema. Unhealthy diets also damage the kidneys and gall bladder, which can cause the delicate skin under the eyes to react with a build-up of fluid and fat.

The maxillary sinus is the cavity just under the eye. The openings, or “ostia”, by which fluid drains from this sinus are located near the roof of the sinus, high on the medial wall. As such, gravity cannot drain the fluid located in the sinus when the head is in an erect position. Over time, a lack of sleep, for example, can cause hydrostatic pressure to slowly lead to the formation of eye-bags. Furthermore, poor fluid drainage can exacerbate temporary causes of inflammation.

It is known to relieve such inflammation of the skin by cooling the skin at the surface. A common method is to apply a cold compress to the area of skin. A reduction in temperature causes blood vessels to constrict, preventing the flow of fluid into soft tissues and reducing the outward pressure on the skin. Static compression enhances this cooling effect due to improved skin contact. Compression also increases the external pressure on the tissue, hindering fluid loss from the vessels and making it more difficult for fluids to accumulate.

A method that is commonly used in respect of facial edema is the application of cucumber slices over the eyes to apply a cooling effect. Similarly, tea bags have been used that have been placed in hot water, then cooled and pressed under the eye. These methods suffer from obvious practical problems, such as a lengthy preparation time, messiness, and the need to hold the compress in place, either manually or using gravity. Accordingly, such methods are generally not used by consumers on a frequent basis.

A further common method of cooling the eye area is an eye-mask. This comprises a gel that is contained within a pack. The gel is cooled by being placed in a fridge, for example, and then the pack is applied to the eye area. These packs are similar to the above methods in that they require a user to hold the pack in place manually, for example by lying down. A further practicality problem is that the packs are generally opaque or translucent, and therefore cannot be used whilst performing a task that requires the user to see.

For individuals that have serious facial edema, there is the option of surgery, called blepharoplasty. This is an invasive operation and, as well as the commonly known risks associated with surgery, can cause an unnatural facial appearance. Also, the cost of such surgery can be substantial.

The art has tried to address the problems identified above.

In particular, WO2010/0191314A1 describes a facial mask for the application of active ingredients such as creams and lotions to the surface of the skin of the face. The mask comprises a an insulator material formed from a non-woven, paper or plastic based material [009,041] to which is applied on the skin contact side of the mask creams and lotions, and a chemical activated cooling element on the other side of the mask [042, 047, 050, 054 and 055], to aid in the action of the creams and lotions. WO2010/0191314A1 also describes as an alternative a vacuum based system to aid with cooling [56 and 57].

WO2007/102362A1 describes an eye cooling sheet comprising a cooling gel (see abstract), wherein the gel is first cooled by placing the sheet in a cooling environment (such as a fridge) prior to its application to the skin of the eye. When the pre-cooled sheet is first placed on the eye there is experienced a heat transfer from the eye to the gel until equilibrium is reached.

CA2135966 describes a device for cooling the surface of the skin via the evaporation of water (page 2 lines 6-13 and page 3 lines 7-14) on an insulating surface such as polyester or cotton (page 4 lines 1-11). Cooling is based on a chemical reaction.

CN201870804U describes an eye cooling system which is based on the cooling effect of cooled water being passed close to the surface of the skin of the eyes. This device comprises inlet and outlet devices that allow the liquid water to flow close to the surface of the skin.

JP10211228A describes an eye cooling system in which the cooling effect is initiated by a cooled powder in an envelope on the device (see figures). In this respect the area around the eye is cooled by conduction of heat from the skin until equilibrium is obtained.

However, the above are either inefficient at cooling the skin surface, or their effects are time limited or transient. Therefore there is a need in the art for devices which have a more efficient cooling mechanism, and devices which are able to maintain their cool profile for extended periods.

Thermoelectric cooling uses the Peltier effect to create a heat flux between the junction of two different types of materials. A Peltier cooler, heater, or thermoelectric heat pump is a solid-state active heat pump, which transfers heat from one side of the device to the other, with consumption of electrical energy, depending on the direction of the current. Such an instrument is also called a Peltier device, Peltier heat pump, solid-state refrigerator, or thermoelectric cooler (TEC). The Peltier device is a heat pump: when direct current runs through it, heat is moved from one side to the other. Therefore it can be used either for heating or for cooling (refrigeration), although in practice the main application is cooling. It can also be used as a temperature controller that either heats or cools. Such devices are commonly used in scientific instruments and computer chips.

Therefore there is a need for a device that can apply a cooling effect to the skin that does not suffer from the above problems. The device described below overcomes the identified problems by the incorporation of active cooling devices by way of heat sink to efficiently transfer the heat from the surface of the skin, thus increasing the efficiency of dynamic cooling of the skin.

The present invention is a portable head-mounted device for cooling the under-eye area of skin. The device comprises at least one cooling element arranged to be located adjacent the skin of the under-eye area when the device is mounted to a head, the cooling element having a first side that faces the under-eye area of skin when the device is mounted to a head and a second side that faces away from the skin of the under-eye area, said second side comprising a heat conductive material. The at least one cooling element comprises a liquid retaining means adapted to retain a liquid provided thereto, and a heat transfer means adapted to transfer heat by conduction to a liquid retained by the liquid retaining means to promote evaporation of said liquid thereby providing a cooling effect when the device is mounted to a head in use.

By “under-eye area” it is meant the periorbital tissue that is below the eye, for example where eye-bags develop. By “portable”, it is meant that the device is light and/or compact enough to be easily carried or moved. By “head-mounted”, it is meant that the device can rest on the head of a wearer without assistance, in a similar fashion to, for example, glasses or goggles. The device is adapted to be worn by the wearer. The device can be a self-contained unit.

In embodiments, two separate cooling elements are used, each corresponding to one of the left and right under-eye areas of skin. In other embodiments, a single cooling element could be used, that for example spans across both the left and right under-eye areas of skin. In accordance with the invention, any number of cooling elements could be used to cool the under-eye areas of one or both the left and right eyes, as long as each cooling element incorporates the liquid retaining means and heat transfer means described herein.

Heat is transferred to the liquid retaining means from the skin via the heat transfer means by conduction. This causes a liquid retained by the liquid retaining means to evaporate. This evaporation causes a cooling effect on the surface of the skin that is in contact with the at least one cooling element, that is the under-eye area. The heat transfer means is important because it enhances the evaporation of a liquid that is retained by the liquid retaining means, over and above simply applying a liquid to the area of skin with, say, a piece of fabric but that does not incorporate a heat transfer means as described herein.

In embodiments the device comprises at least one reservoir for holding a liquid to be supplied to the liquid retaining means, wherein said at least one reservoir is in fluid communication with said at least one cooling element for supplying liquid to the liquid retaining means. The reservoir may be in fluid communication with the liquid retaining means. By “fluid communication”, it is meant that the reservoir is arranged such that liquid held therein may be supplied to the liquid retaining means. Thus a liquid that is brought into contact with the under-eye area of skin by said liquid retaining means is at least partially sourced from the reservoir. Using a reservoir in this way allows the device to have a constant and replenishable source of liquid for bringing into contact with the skin. This is especially important when used in conjunction with said heat transfer means, because in use the liquid will be evaporating at a fast rate and will need to be replenished constantly. A reservoir therefore prolongs the use of the device.

The reservoir may be integral with the device, for example a cavity or chamber in the device that can be filled with liquid, for example as described below in relation to the preferred embodiment. Alternatively, the reservoir may comprise a separate removable and re-attachable reservoir, for example a liquid-holding vial. “Vial” is intended to mean a small container that is adapted to hold liquid. The re-attachable reservoir may attach or be inserted onto or into the device in such a way that a liquid held therein can be supplied to the liquid retaining means of the at least one cooling element. Using an attachable and/or removable reservoir would provide the advantage of being able to easily replenish the liquid supplied to the liquid retaining means, that is without needing to manually apply liquid to the liquid retaining means, or to a reservoir that is integral with the device e.g. in the form of a cavity in the device. The invention extends to a device which does not include such a separate reservoir e.g. vial but is configured for attachment to such a reservoir. The invention also extends to a device configured for attachment to a separate reservoir e.g. vial for holding a liquid to be supplied to the liquid retaining means.

Alternatively, in other embodiments, the device does not comprise a reservoir of fluid and is not configured for attachment to a reservoir. In such embodiments, liquid can be applied to the liquid retaining means directly using any kind of applicator known in the art, for example a pipette.

In accordance with some preferred embodiments, the at least one cooling element comprises two distinct components, wherein the first component comprises or constitutes said liquid retaining means and the second component comprises or constitutes said heat transfer means. The first and second components may be distinct or different from one another, as described below, and preferably comprise different materials.

In accordance with the invention in any of its aspects or embodiments the liquid retaining means may comprise or be a material that is capable of taking up liquid by capillary action or wicking, such as an absorbent material. In some embodiments also involving a reservoir as described above, a liquid held in the reservoir may be wicked from the reservoir by the liquid retaining means. For example, the liquid retaining means can be at least partially submerged or otherwise in contact with a liquid held in the reservoir.

The heat transfer means may comprise or be a highly thermally conductive material, such as a metal. Particularly suitable metals are those having a high thermal conductivity, for example copper, aluminium, silver and gold. Using a highly thermally conductive material rapidly transfers heat to the liquid and enhances the cooling effect. In some embodiments, at least part of the heat transfer means is arranged to contact the under-eye area of skin when worn. The heat transfer means may comprise a skin contacting surface. The heat transfer means is adapted to transfer heat by conduction to a liquid retained by the liquid retaining means. The heat may be heat from the surrounding environment and/or the skin, depending on whether the heat transfer means is arranged to contact the skin.

It will be appreciated that either of the liquid retaining means or the heat transfer means may define at least part or all of a first skin contacting surface of the device. The liquid retaining means and the heat transfer means may be on either side of one another or may be intermixed.

In preferred embodiments the cooling element is arranged such that a liquid retained by said liquid retaining means comes into contact with the skin of the under-eye area when the device is worn in use. For example this could be due to the cooling element being in close proximity to, or in contact with the under-eye area of skin.

In some embodiments, the liquid retaining means is arranged to contact the under-eye area of skin when the device is worn to bring liquid into contact with the under-eye area of skin. For example, if the liquid retaining means comprises an absorbent material, then at least some of the material preferably contacts the under-eye area of skin in use. In one embodiment, the heat transfer means is a metal mesh that at least partially encloses the liquid retaining means. In some embodiments the liquid retaining means comprises an absorbent material, and the fibres of the absorbent material protrude through the mesh. In another such embodiment, the liquid retaining means and heat transfer means may be intermixed with one another, for example a metal grid may be contained within the fibres of an absorbent material. It will be appreciated that the liquid retaining means and the heat transfer means will still be distinguishable from one another.

It will be appreciated that the liquid retaining means may not be arranged to directly contact the skin. A liquid retained therein can be brought into contact with the under-eye area of skin via an intermediate component, for example the heat transfer means. The heat transfer means may be between the liquid retaining means and the skin. For example, the heat transfer means could comprise or be a layer of metal that contacts the under-eye area of skin when the device is worn, and the liquid retaining means could comprise or be a layer of absorbent material adjacent to this layer of metal, that is toward the second side of the device. In embodiments in which the liquid retaining means does not directly contact the skin, the cooling element should be suitably arranged such that liquid may be transferred to the skin, for example through openings in the heat transfer means when the heat transfer means is located between the skin and the liquid retaining means in use. Liquid retained by the liquid retaining means should be in fluid communication with the first side of the cooling element(s).

It is advantageous to increase the contact area between the liquid retaining means and the heat transfer means, to enhance evaporation of a liquid retained by the liquid retaining means as much as possible, and thus improve the cooling effect.

In embodiments the portable head-mounted device is adapted to secure the at least one cooling element to the under-eye area of skin. The device may comprise support means adapted to secure said at least one cooling element against the under-eye area. The support means for the device preferably holds or presses the cooling element(s) against the under-eye area, and may comprise a substantially rigid frame preferably similar in construction to a glasses frame. However, it is within the scope of the invention that one or more flexible bands can be provided to support the cooling element(s) instead. For example, one or more flexible bands could be adapted to support the cooling elements, and wrap around the ears, or the head, of a wearer in use. A support means could also be provided that comprises in part a rigid frame as well as one or more flexible bands. For example, the cooling element(s) could be held by a rigid portion of the support, whilst one or more flexible bands could attach to the rigid portions of the support to wrap around the ears or head of a wearer in use, thereby securing the cooling element(s) against the under-eye area. Whatever means is used to support the cooling element(s), it must be such that the device is portable, as defined above. This makes the device easy to use on a day-to-day basis.

In some embodiments, the support means comprises a front portion for supporting said at least one cooling element. The front portion comprises first and second side portions either side of a bridge portion, wherein said bridge portion is adapted to rest on the nose of a wearer in use. The first and second side portions support first and second cooling elements corresponding to respective right and left under-eye areas of skin. First and second arms may attach to respective first and second side portions, said arms resting on the ears of a wearer, or configured to hold against the side of a wearer's head in use, in a similar fashion to glasses. In these embodiments, the front portion, bridge portion and first and second arms are preferably rigid.

A reservoir for containing liquid as described above may be contained within the support means, for example held in a cavity or chamber within the support means. In a preferred embodiment, a reservoir is contained in the first and second side portions, and as described above the first component, that is the liquid retaining means, is at least partially submerged or otherwise in contact with a liquid held in the reservoir. Alternatively, an intermediate channel or conduit could be provided that provides fluid communication between the reservoir and the liquid retaining means. For example, a reservoir could comprise a removable vial as described above that attaches or connects to the device, and from which fluid contained therein drains and is fed to the liquid retaining means via one or more channels or conduits.

The at least one cooling element is preferably configured so as to only contact the under-eye area of skin of a wearer when the device is mounted to a head in use. That is, the cooling element(s) are not configured in such a way as to contact the eyes or eye-lids, and block or significantly impair the eyesight of a user wearing the device. Put another way, the cooling elements do not extend substantially into the eye area when the device is mounted to the head in use, thereby substantially impairing or blocking the vision of a wearer. This means that a user wearing the device can perform normal functions such as walking or reading whilst wearing the device.

In preferred embodiments the device comprises a thermally conductive arrangement located on the second side of the at least one cooling element and in thermal communication with said heat transfer means of said cooling element. In some preferred embodiments, the thermally conductive component comprises one or more fins in thermal communication with the heat transfer means. The thermally conductive arrangement may contact the heat transfer means. This increases the surface area of the portion of the device that transfers heat to a liquid retained by the liquid retaining means, which enhances the evaporation of the liquid. The thermally conductive component may be a metal, for example copper, aluminium, silver or gold. The thermally conductive component may comprise the same material as the heat transfer means.

A liquid may be provided in combination with the device. The liquid may be a topical medication, for example an antioxidant or astringent. The liquid may contain at least one of: tretinoin; retinoids; alpha-hydroxy acids; estrogens; vitamin C; vitamin C derivatives; anhydrous vitamin C combo; vitamin E; ferulic acid.

The present invention extends to the use of a device in accordance with any of the embodiments of the invention to cool the under-eye area and a method of cooling the under-eye area using the device. The method may comprise mounting the device to the head of a wearer with the at least one cooling element adjacent the under-eye area. The method may comprise applying liquid to the liquid retaining means.

Various embodiments of the present invention will now be described, by way of example only, and with reference to the following drawings, in which:

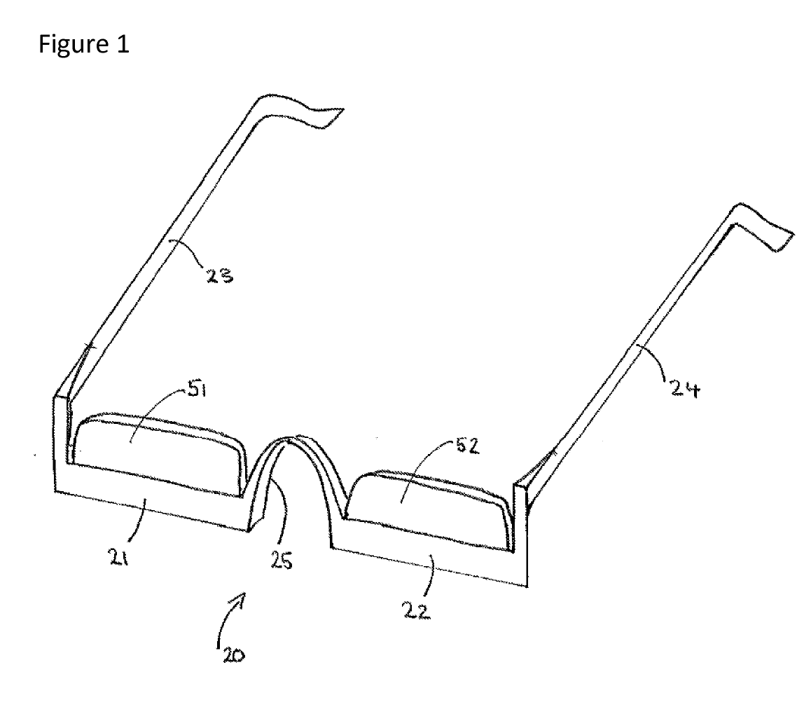

FIG. 1 is a schematic view of a first embodiment of the invention.

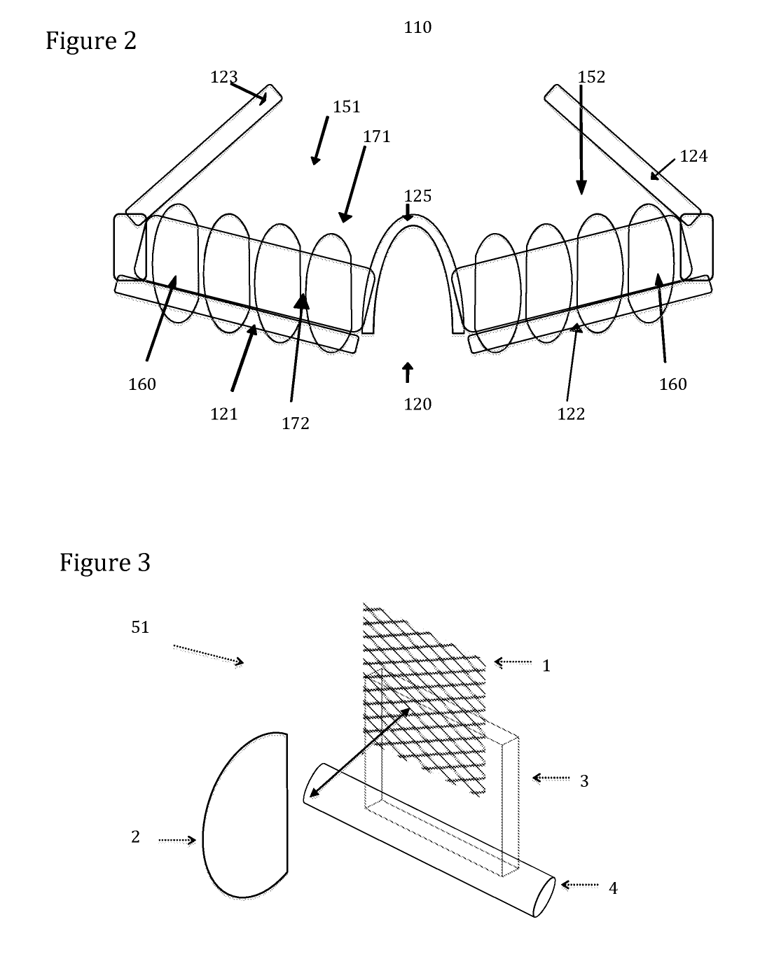

FIG. 2 is a schematic view of a second embodiment of the invention.

FIG. 3 is a schematic view showing in more detail the construction of a cooling element and related components of the device, useable with either of the first or second embodiments of the invention.

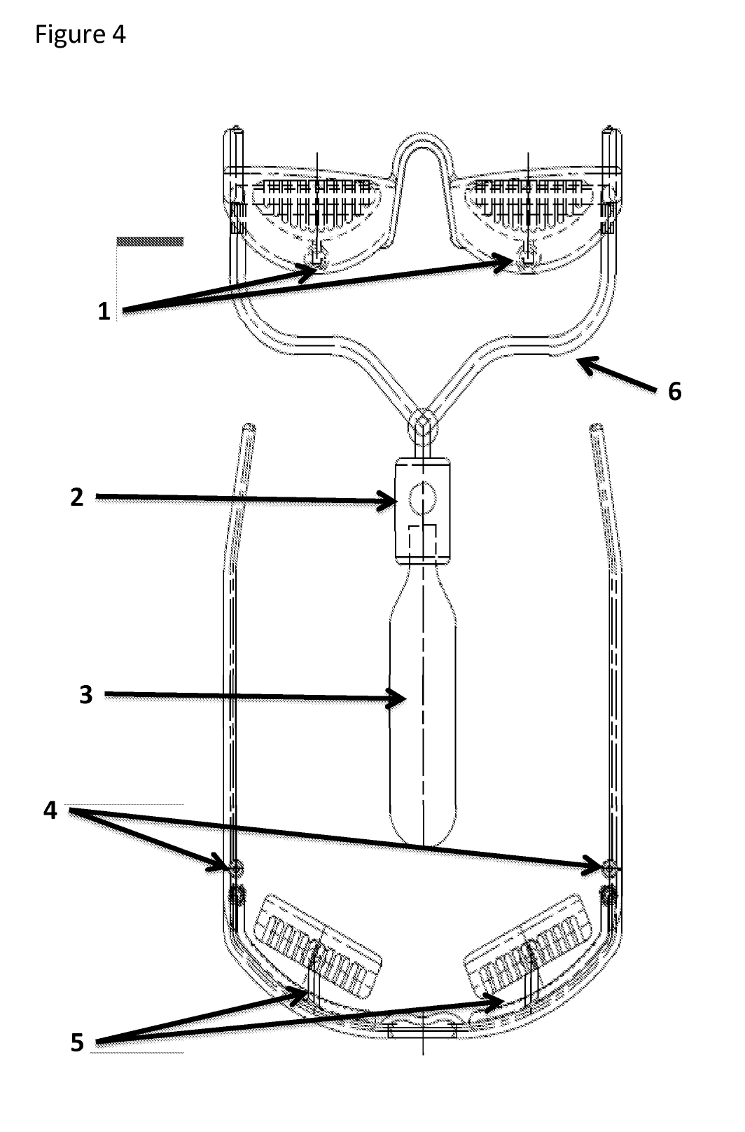

FIG. 4 is a schematic of the preferred embodiments of FIGS. 1 to 3 illustrating a forced convection system. In the figure, portion 1 shows the eyepiece with rotational adjustment via friction fit ball joint in vertical and horizontal plane to fit face below eye socket. Portion 2 shows a Flow Control Valve, and portion 3 shows a compressed air supply refillable via pump or receptacle. Portion 4 shows pivots for fitting of the device to the face and folding for carriage. Portion 5 shows the directional air vents pointing at the eye-piece heat sink and portion 6 shows a flexible tube.

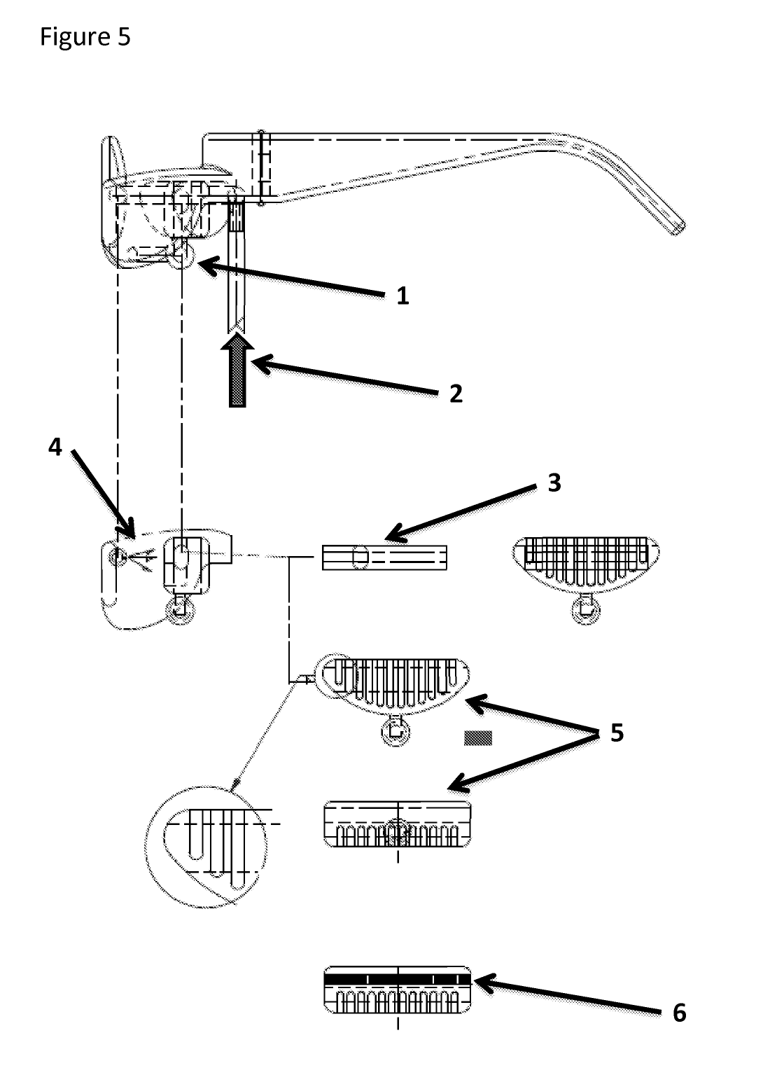

FIG. 5 show additional drawings of the most preferred embodiment illustrated in FIG. 4 where 1 shows a ball joint, the arrow 2 indicates the direction of air flow, 3 shows wadding for the reservoir, 4 shows the direction of the forced air-flow, 5 shows the eye piece heat-sink with the water held in the slots by capillary action. Portion 6 relates to a 2^{nd} embodiment wherein a Peltier chip is sandwiched within the heat sink to control the degree of cooling effect.

FIG. 1 illustrates a preferred embodiment of the device 10. The device 10 is similar to a pair of glasses. A front portion 20 of the device 10 comprises a bridge portion 25 that separates first and second side portions 21 and 22 respectively. First side portion 21 corresponds to the right side of the face, and the right eye. Second side portion 22 corresponds to the left side of the face, and the left eye. The front portion 20 and first and second side portions 21 and 22 may be integral with one another as shown. The device 10 further comprises first and second arms, 23 and 24 respectively, that extend from the first and second side portions 21 and 22, respectively. First and second arms 23 and 24 may be attached to first and second side portions 21 and 22 using any type of mechanism known in the art, for example a hinge mechanism.

The device 10 further comprises first and second cooling elements, 51 and 52 respectively. When the device 10 is worn in use, first cooling element 51 is held or pressed against the wearer's right under-eye area of skin, and second cooling element 52 is held or pressed against the wearer's left under-eye area of skin. In the illustrated embodiment, the first and second cooling elements 51 and 52 are configured so as to only contact the under-eye area of skin. That is, the cooling elements do not extend substantially into the eye area when the device is mounted to the head in use, thereby substantially impairing or blocking the vision of a wearer in use. It will be appreciated that the person skilled in the art will know how to configure the device 10 in such a way, so as to only contact the under-eye area, and not substantially impair the vision of a wearer in use.

The device 10 may comprise first and second reservoirs that are in the form of a cavity or chamber in the first and second side portions 21 and 22 of the device 10. The first and second cooling elements 51 and 52 may then extend at least partially into the reservoir to contact a liquid held therein, as illustrated below in FIG. 3. Although in the preferred embodiment the reservoir is in first and second side portions 21 and 22 of the device 10, in other embodiments the reservoir can be in any portion of the device 10, as long as the reservoir is in fluid communication with the cooling element(s) as described above. A reservoir can provide the device 10 with a constant source of liquid, to allow prolonged use of the device 10. For example, if the cooling elements 51 and 52 of the device comprise a first component in the form of an absorbent material as described above, then in use this material will continually absorb liquid by wicking the liquid held in the reservoir by capillary action.

First and second arms 23 and 24 may be configured to rest on the right and left ears of the wearer in use as illustrated. Alternatively, first and second arms 23 and 24 may be configured to hold against the side of a wearer's head in use. The bridge portion 20 is configured such that at least part of the bridge portion 20 rests on the wearer's nose in use. The device 10 therefore rests on the head of a wearer without assistance. For example, the device does not have to be held in place by the wearer, and does not require the user to be lying down. The device does not therefore interfere with the ability of a user to go about their normal activities.

FIG. 2 illustrates another embodiment of the device 110. The device 10 is similar to that shown in FIG. 1, and comprises a front portion 120 comprising a bridge portion 125 separating first and second side portions 121 and 122, first and second arms 123 and 124, first and second cooling elements 151 and 152. The cooling elements 151 and 152 comprise a first side 171 and an opposite second side 172. The first side 171 faces the under-eye area of skin of a wearer when the device is mounted to a head in use, whilst the second side 172 faces away from the under-eye area. The device 10 comprises a thermally conductive arrangement 160 attached to said second side 172 of the cooling elements 151 and 152. In the illustrated embodiment, the thermally conductive arrangement 160 is in the form of one or more fins 161. The thermally conductive arrangement 160 is in contact with the heat transfer means of the cooling elements 151 and 152. The fins increase the surface area of the conductive material present for conducting heat away from the under-eye area.

In preferred embodiments, the cooling elements 51, 52 or 151, 152 comprise liquid retaining means in the form of a first component and heat transfer means in the form of a second distinct or different component. An example of such a cooling element is illustrated schematically in FIG. 3. This may be used in either of the illustrated embodiments of the invention shown in FIGS. 1 or 2. The first component comprises an absorbent material 3, that is a material that is capable of absorbing liquid, for example a sponge. The second component comprises a metal sheath 1 that contacts or surrounds the absorbent material 3. The sheath 1 has gaps or openings in it that allow the absorbent material 3 to protrude through and contact the under-eye area of skin in use. In this way, liquid that is retained by the absorbent material 3 is brought into contact with the under-eye area of skin. The metal sheath 1 facilitates evaporation of the liquid from the absorbent material 3 by conducting heat from the skin and transferring this to the absorbent material and any liquid retained therein. The second component may be silver. Silver has a very high thermal conductivity, which enhances the conduction of heat to the liquid in the first component.

FIG. 3 also shows an example of a metal fin 2 that contacts the metal sheath 1. The fin 2 provides a means for heat to be conducted away from the metal sheath 1. The enlarged surface area provided by the fin 2 increases the rate of heat transfer. In embodiments in accordance with FIG. 1, such fins may be omitted.

The cooling element of FIG. 3 is located such that the absorbent material 3 is partially submerged in the liquid that is held in a reservoir of the device 10, 110. In this way, liquid is wicked from the reservoir 4 into the absorbent material 3 by capillary action.

To use the device 10, 110 a person attached or places the device 10, 110 on their head, such that the at least one cooling element 51, 52 or 151, 152 contacts the under-eye area of skin. Liquid is supplied to the liquid retaining means. This can be directly, for example using a pipette to apply liquid to a liquid retaining means in the form of an absorbent material, or indirectly, for example attaching a vial of liquid to the device 10, 110 wherein liquid initially held in the vial is fed to the liquid retaining means, for example via a channel or conduit. The liquid is then brought into contact with the skin. Also, the liquid evaporates from the liquid retaining means to cool the skin.

In embodiments in which the device incorporates an integral reservoir as shown in FIG. 3, liquid is initially held in the reservoir 4 and is wicked from the reservoir 4 into the absorbent material 3 by capillary action. In some embodiments, the absorbent material may protrude through the metal sheath and directly contact with the skin. Liquid is thus brought into contact with the under-eye area of skin by virtue of this direct contact. In embodiments in which the absorbent is not in direct contact with the skin, liquid can be brought into contact with the under-eye area of skin via the metal sheath. That is, the liquid travels over the metal sheath and contacts the under-eye area of skin. In embodiments in which the liquid is a topical medication, the under-eye area of skin will thus be treated by the liquid. The metal sheath 1 is in contact with the skin, and transfers heat from the skin to the liquid by conduction. The liquid evaporates, producing a cooling effect that cools the under-eye area of skin. The evaporation is enhanced by the action of the metal sheath which transfers heat by conduction to the liquid. In embodiments incorporating a reservoir, upon evaporation of the liquid from the absorbent material 3, more liquid is wicked from the reservoir by said capillary action, and the process starts again.

In a preferred optional embodiment forced convection can be employed to improve cooling. Forced convection is a mechanism, or type of transport in which fluid motion is generated by an external source (like a pump, compressed air, mini-blow, fan, suction device, etc.).

In the most preferred embodiment the device incorporates a thermoelectric heating/cooling element such as a Peltier device (6) and associated power supply such as for example a battery. The thermoelectric heating (6) and cooling device is preferably embedded in, or in contact with the heat sink (5). Advantageously such a device when in association with the heat sink (5) will aid in the modulation of heat transfer from the surface of the skin. Optionally, such a heat sink/thermoelectric heating cooling device can be used in association with the liquid cooling system (as described above) to increase the rate of cooling of the system. Control of the thermoelectric heating and cooling device is achieved by modulating the current of the device.

In addition to the above such a device may also be used to actively increase the dynamic range of the heating or cooling effect of the heat sink. Thus this device differs from the prior art.

The following descriptions relate to differences to prior art stated on page 2 above: WO2010/0191314A1 (Young); WO2007/102362A1 (Kobayashi); CA2135966 (Lavoie); CN201870804U; (Li); JP10211228A (Unity).

Young achieves evaporation through the creation of a vacuum environment, i.e. pressure, by contrast our means of heat exchange uses the efficient thermal conduction properties of the metal heat sink increase the temperature of water, and thus facilitate evaporation. Also Young's device does not dissipate the heat as the energy is still contained in the system. This mask contains no medium through which energy can be conducted from the users face into the said water particles to give rise to a cooling effect. Indeed a vacuum encapsulated material is categorically the worst form of heat conductor and is thus used in industry as heat insulation. The result being no cooling effect.

The device of this application releases the evaporated heat energy into the environment and away from the user. In addition the device applies targeted pressure for the purpose of supplying the delicate skin under the eye with support to counter the hydrostatic pressure caused by the accumulation of fluid. Young's facemask covers the whole face with no intention to apply targeted pressure.

The cooling effect of Young's product is designed to treat Rosaca with generalised cooling of various areas including under the eyes. However, as stated, the method of cooling is different, as is the application of pressure. Our product has the advantage of having a sustained cooling effect, with the precise application of pressure to treat a specific condition. The cooling effect of the device is designed to treat peri-orbital edema and thus application of pressure to counter the hydrostatic pressure and fluid accumulation under the eyes, in conjunction to said cooling effect.

Kobayashi describes a product that cools the eye by means of a gel. This is known in the art and is achieved through decreasing the temperature of the gel, through the use of a fridge or freezer, thus causing a temperature gradient and inducing thermal transfer away from the eye. This does indeed produce a cooling effect, however the area of cooling is described as being over the eye itself, and not under the eye as in our product. Their device cools the eyeball, and requires the user to have their eyes closed during treatment. This device uses a different method for inducing a cooling effect, and is specifically to treat edema under the eye. Therefore the area of treatment is different, as is the condition we are treating. Furthermore, Kobayashi's product is attached to the eyebrow, specifically the upper part of the eye socket, and held in place with a form of adhesive. Our product does not contact the eyebrow area nor is it held in place with adhesive.

One of the differences between this device and Lavoie's are firstly pertaining to the materials. They use a permeable envelope containing a material with high absorption/desorption properties held together using a permeable envelope made of cotton/polyester blend. This is known in the art, but their patent relates to the structure forming a band, which can be wrapped around the head. Potentially this can be used to cover the eyes, however it is not designed specially for this purpose therefore there is no targeted pressure under the eyes. Also, the materials we use are primarily metallic and thus we are using the properties of said metals for their high thermal conductivity. This is absent in Lavoie's product. Another difference with our device described above, relating to structure, is the inclusion of fins to increase surface area and thus heat transfer to the surroundings.

Li's device relates of the circulation of cold water around the circumference of eye. Firstly, cold water is pumped through said inlet and outlet, however the method of attaining cool water is unknown. Perhaps this is attached to a system of pre-cooled water, in which case the cooling effect is created through the use of a fridge or freezer. Firstly, our device is different in that we use evaporative cooling. Secondly, our device is designed in order to provide targeted pressure under the eye for the specific treatment of peri-orbital edema. The purpose of Li's product is to treat “eye injury”. We do not use plastics, as in Li's product, due to their poor thermal conductivity. Lastly, we do not require a mechanical pump as in Li's model, as our product utilises natural capillary action in order for water to envelop the system in readiness for evaporation.

Unity's product achieves a cooling effect through a powdered substance, which is said to be a “cooling agent”. It is unclear what this substance is or whether there is an endothermic reaction, or whether the powder is pre-cooled in a fridge/freezer. In any scenario, their method of cooling is not evaporative cooling and thus is different to our own product. A second difference is that their eye-piece is opaque and designed to be worn over the whole of the eye area. Our device allows complete visibility giving the user the ability to continue to work and be productive during treatment. A third difference is Unity's product does not specifically treat peri-orbital edema, and so there is no pressure to counter the hydrostatic pressure of said edema.

Although the present invention has been described with reference to preferred embodiments, it will be understood by those skilled in the art that various changes in form and detail may be made without departing from the scope of the invention as set forth in the accompanying claims.

1. A portable head-mountable device for cooling the under-eye area of skin, said device comprising at least one cooling element arranged to be located adjacent the skin of the under-eye area when the device is mounted to the head, the cooling element having a first side configured to faces the under-eye area of skin when the device is mounted to a head and a second side configured to faces away from the skin of the under-eye area;
said at least one cooling element comprising:
liquid retaining means adapted to retain a liquid provided thereto; and
heat transfer means adapted to transfer heat by conduction to a liquid retained by the liquid retaining means to promote evaporation of said liquid thereby providing a cooling effect when the device is mounted to the head in use.

2. The device of claim 1 wherein the cooling element is arranged such that a liquid retained by said liquid retaining means may come into contact with the skin of the under-eye area when the device is worn in use.

3. The device as claimed in claim 1, further comprising at least one reservoir for holding a liquid to be supplied to the liquid retaining means, wherein said at least one reservoir is in fluid communication with said at least one cooling element for supplying liquid to the liquid retaining means.

4. The device as claimed in claim 1, wherein said at least one cooling element comprises two distinct components, wherein a first component comprises or constitutes said liquid retaining means, and a second component comprises or constitutes said heat transfer means.

5. The device as claimed in claim 1, wherein said liquid retaining means comprises a material capable of taking up liquid by capillary action or wicking.

6. The device as claimed in claim 1, wherein said liquid retaining means comprises an absorbent material.

7. The device as claimed in claim 1, wherein said heat transfer means comprises a thermally conductive material.

8. The device as claimed in claim 1, wherein said heat transfer means comprises a metal.

9. The device as claimed in claim 8, wherein said metal comprises at least one of copper, aluminium, silver and gold.

10. The device as claimed in claim 1, wherein said heat transfer means is in the form of a metal mesh or lattice structure, and said liquid retaining means is adjacent to said metal mesh or lattice structure.

11. The device as claimed in claim 1, wherein said at least one cooling element is layered, and the liquid retaining means and heat transfer means form respective ones of the layers.

12. The device as claimed in claim 1, wherein fluid flow is facilitated with forced convection.

13. The device as claimed in claim 12, wherein forced convection is facilitated by a mini-blower, a pump or compressed air.

14. The device as claimed in claim 1, wherein the device further comprises a thermoelectric heating/cooling element (6) and associated power source, characterised in that the thermoelectric heating/cooling element is embedded in, or contacted with a heat sink (5).

15. The device as claimed in claim 14, wherein the thermoelectric heating/cooling element is a peltier device.

16. The device as claimed in claim 1, further comprising support means adapted to mount the device to the head of a wearer in use and secure said at least one cooling element against the under-eye area.

17. The device as claimed in claim 16, wherein said support means comprises at least one flexible band.

18. The device as claimed in claim 16, wherein said support means comprises a substantially rigid frame

19. The device as claimed in claim 16, wherein:
said support means comprises a front portion having first and second side portions separated by a bridge portion;
said bridge portion is adapted to rest on the nose of a wearer when the device is mounted to a head in use;
said at least one cooling element comprises a first cooling element and a second cooling element; and
said first cooling element is supported by said first side portion and said second cooling element is supported by said second side portion.

20. The device as claimed in claim 16, wherein said support means is adapted to hold or press said at least one cooling element against the under-eye area of skin of a wearer when the device is mounted to a head in use.

21. (canceled)

22. (canceled)

23. (canceled)
